(19) United States
(12) Patent Application Publication
(10) Pub. No.: US 2014/0277005 A1
(43) Pub. Date: Sep. 18, 2014

(54) MEDICAL DEVICE INCLUDING FLEXIBLE ELONGATE TORQUE-TRANSMITTING MEMBER

(71) Applicant: Covidien LP, (US)

(72) Inventor: Ethan A. Guggenheimer, Minneapolis, MN (US)

(73) Assignee: COVIDIEN LP, Mansfield, MA (US)

(21) Appl. No.: 13/804,999

(22) Filed: Mar. 14, 2013

(51) Int. Cl. A61B 17/3207

(52) U.S. Cl. CPC A61B 17/320758
USPC 606/159

(57) ABSTRACT

The present disclosure relates to a catheter including a torque-transmitting member operatively connected to a functional element of the catheter. The torque-transmitting member may include a flexible structural core, an inner radial layer, and an outer radial layer. The structural core has a plurality of adjacent core segments that move away from one another when the torque-transmitting member is bent along an arc. The inner radial layer has an inner surface secured to the plurality of core segments such that the inner layer experiences localized tensile strain when the core segments at the outer radius of the arc are urged away from one another during bending of the torque-transmitting member. The inner radial layer may comprise an elastomer.

### FIELD OF THE DISCLOSURE

The present invention generally relates to a medical device including a flexible elongate torque-transmitting member.

### BACKGROUND

Medical diagnostic and treatment catheters may include a flexible elongate torque-transmitting member received in a body of the catheter to impart rotation to a functional element of the catheter. For example, debulking catheters, such as atherectomy and thrombectomy catheters, include a material-removing element, such as a cutting blade, that is rotated by a flexible driveshaft to remove material (e.g., tissue) from a body lumen of a subject.

Typically, the driveshaft of such a catheter has a structural driveshaft core including helical or coiled metal wires extending along a length of the driveshaft, and a polymer laminate circumferentially surrounding the driveshaft core. During some diagnostic and treatment procedures, the catheter is inserted in a tortuous path of a body lumen so that the catheter, and the driveshaft, is bent along an arc having a small radius of curvature. In such a case, the internal torsional resistance of the driveshaft may increase, which may negatively impact the performance of the catheter.

### SUMMARY

In one aspect, the present disclosure is related to a catheter including a torque-transmitting member operatively connected to a functional element of the catheter. The torque-transmitting member may include a flexible structural core, an inner radial layer, and an outer radial layer. The structural core has a plurality of adjacent core segments that move away from one another when the torque-transmitting member is bent along an arc. The inner radial layer has an inner surface secured to the plurality of core segments such that the inner layer experiences localized tensile strain when the core segments at the outer radius of the arc are urged away from one another during bending of the torque-transmitting member. The inner radial layer may comprise an elastomer. The driveshaft may have an internal torsional resistance from about 0.010 in-oz (0.007 N-cm) to about 0.10 oz-in (0.07 N-cm) when bent along an arc having a radius of curvature of about 0.5 in (1.27 cm). The outer layer may be heat shrunk around the inner layer and/or may have a low modulus of elasticity.

Other objects and features will be in part apparent and in part pointed out hereinafter.

### BRIEF DESCRIPTION OF THE DRAWINGS

FIG. 1 is a fragmentary perspective of a catheter assembly including a catheter and a removable handle attached to the catheter;

FIG. 2 is an enlarged, fragmentary longitudinal sectional view of the catheter with a cutting element in a stored position;

FIG. 3 is similar to FIG. 2, except with the cutting element in the deployed or cutting position;

FIG. 4 is an enlarged, partial side elevational view of a driveshaft of the catheter in a straight or linear configuration;

FIG. 5 is similar to FIG. 4, except the driveshaft is bent along an arc;

FIG. 6 is a cross section of the driveshaft;

FIG. 7 is a side elevational view of a core of the driveshaft, showing fragmentary filar layers including helical strands wrapped around a longitudinal axis of the driveshaft;

FIG. 8 is a schematic cross section of adjacent strands of a driveshaft core and a corresponding bridge portion of an inner driveshaft layer at a top portion of the driveshaft in FIG. 4;

FIG. 9 is a schematic cross section of adjacent strands of the driveshaft core and a corresponding bridge portion of the inner driveshaft layer at a bottom portion of the driveshaft in FIG. 4;

FIG. 10 is a schematic cross section of the adjacent strands and corresponding bridge portion of the inner driveshaft layer at the top portion or outer radius of the arc along which the driveshaft in FIG. 5 is bent;

FIG. 11 is a schematic cross section of the adjacent strands and corresponding bridge portion of the inner driveshaft layer at the bottom portion of inner radius of the arc along which the driveshaft in FIG. 5 is bent;

FIG. 12 is a line graph for determining an elastomeric limit or elastic yield strain of PEBAX® materials;

FIG. 13 is a perspective of a Torque Resistance Testing Apparatus used to measure internal torque resistance of drive shafts;

FIG. 14 is an enlarged, fragmentary front view of a chuck and a plate of the Torque Resistance Testing Apparatus; and

FIG. 15 is a line graph of data collected testing drive shafts.

Corresponding reference characters indicate corresponding parts throughout the drawings.

### DETAILED DESCRIPTION OF THE DRAWINGS

The present disclosure relates to a flexible elongate torque-transmitting member for a functional element of a medical device, and in one example, to a flexible elongate torque-transmitting member received in a lumen of a body of a catheter. Non-limiting examples of medical devices in which the torque-transmitting member may be incorporated in a body of a catheter, according to the principles of the present disclosure, include debulking catheters, including debulking catheters having a material-removing element (broadly, a functional element) rotated by the torque-transmitting member to remove material (e.g., tissue) from a body lumen of a subject; catheter and/or guidewire systems for treating chronic total occlusions; and visualization catheters having an imaging device, such as a camera, ultrasound transducer, etc. (broadly, a functional element) rotated by the torque-transmitting member. The torque-transmitting member may be incorporated in other types of medical devices without departing from the scope of the present invention.

Referring to FIG. 1, an embodiment of a debulking catheter for removing material (e.g., tissue) from a body lumen (e.g., a blood vessel) is generally indicated at 10. The catheter 10 includes an elongate, generally flexible body, generally indicated at 12, having proximal and distal ends 12a, 12b and a length extending between the proximal and distal ends. The body 12 defines a longitudinal lumen 16 (see also, FIGS. 2 and 3) extending along the length of the body. The body 12 has a proximal body portion 14a and a distal body portion 14b pivotally secured to the proximal portion at a pivot axis P. The body 12 (e.g., the proximal body portion) may include a torque tube for transmitting rotation of the proximal end 12a of the body toward the distal end 12b of the body. A generally flexible driveshaft 20 (broadly, a torque-transmitting member) is received in the body lumen 16 and extends from the proximal end 12a toward the distal end 12b of the body 12. The driveshaft 20 is described in more detail below. A tissue-removing element, generally indicated at 24 (broadly, a functional element), for removing plaque from a blood vessel is fixedly secured to the distal end of the driveshaft 20 so that rotation of the driveshaft about its longitudinal axis imparts conjoint rotation of the tissue-removing element. The distal body portion 14b may include a tissue containment chamber (indicated at 25 in FIGS. 2 and 3) for receiving tissue removed from the body lumen by the tissue-removing element 24. Other configurations of the body 12 may be used, including for example, one which does not have a tissue containment chamber at its distal end.

Referring still to FIG. 1, a handle, generally indicated at 26, is removably attached to the proximal end 12a of the catheter body 12 for use in operating the catheter 10. The handle 26 includes a motor 28 that is operatively connected to a proximal portion of the driveshaft 20 for driving rotation of the driveshaft and the tissue-removing element 24. A power source 30, such as batteries, powers the motor 28. An actuator 34 (e.g., a slide) of the handle 26 is mechanically connected to the driveshaft 20. As explained in more detail below, the actuator 34 allows the user to longitudinally move the driveshaft 20 within the body lumen 16 of the catheter body 12 to move the tissue-removing element 24 between a stored position, in which the tissue-removing element is received in the catheter body, and a deployed position, in which the tissue-removing element extends outside the body to engage and remove tissue. The actuator 34 is also connected to a switch (not shown) for activating the motor 28 when the tissue-removing element 24 is deployed, and deactivating the motor 28 when the tissue-removing element is stored in the catheter body 12. It is understood that the handle 26 may be of other configurations without departing from the scope of the present invention.

Referring to FIGS. 2 and 3, in the illustrated embodiment, the tissue-removing element 24 comprises a cutting element having a cutting edge 36 facing distally, although in other embodiments the cutting edge may face proximally, and a cup-shaped surface 37 for directing removed tissue distally into the tissue containment chamber 25. In the deployed position shown in FIG. 3, the cutting element 24 extends partially outside a window 38 defined by the catheter body 12. In the stored position shown in FIG. 2, the cutting element 24 is disposed within the catheter body 12 and does not extend outside the window 38. The illustrated embodiment includes a deployment mechanism. The deployment mechanism includes the cutting element 24, which functions as a cam or camming element, the distal body portion 14b (e.g., a cutting element housing) that is pivotal relative to the proximal body portion 14a and at least partially defines the window 38, and ramp or cam follower 40 fixedly secured inside the distal body portion.

As set forth above, the cutting element 24 is moveable longitudinally within the catheter body 12. Accordingly, as the cutting element 24 moves longitudinally within the body 12, it engages and moves longitudinally along the cam follower 40, causing the distal body portion 14b to pivot relative to the proximal body portion 14a of the catheter body 12 about the pivot axis P. In particular, in the illustrated embodiment moving the driveshaft 20 proximally, such as by moving the actuator 34 proximally, imparts proximal movement of the cutting element 24 along the cam follower 40, which causes the distal body portion 14b to pivot or deflect away from the proximal body portion 14a so that the cutting element 24 extends out the window 38. Moving the driveshaft 20 distally, such as by moving the actuator 34 distally, imparts distal movement of the cutting element 24 along the cam follower 40, which causes the distal body portion 14b to pivot or deflect toward the proximal body portion 14a so that the cutting element 24 is received in the cutting element housing 39. A suitable deployment mechanism for moving the cutting element 24 between its deployed and stored positions is disclosed in U.S. patent application Ser. No. 11/012,876, filed Dec. 14, 2004, which is hereby incorporated by reference in its entirety. It is understood that a catheter constructed according to the principles of the present disclosure may not include a deployment mechanism (e.g., the tissue-removing element or other functional element may always be deployed or may remain within the catheter body). The tissue-removing element 24 may comprise other devices, other than the illustrated cutting element, in other embodiments. For example, the tissue-removing element may comprise an abrasive element having an abrasive surface. Other tissue-removing elements do not depart from the scope of the present invention.

Referring to FIG. 6, in the illustrated embodiment the driveshaft 20 comprises a flexible driveshaft core, generally indicated at 46, including at least one filar layer of at least one strand 48 (e.g., wire or thread) that is helically wound (or coiled) around a longitudinal axis LA of the driveshaft to form a helix; an inner radial layer 50 circumferentially surrounding the driveshaft core; and an outer radial layer 52 circumferentially surrounding the inner radial layer. The inner radial layer 50 is secured to (i.e., bonded or adhered to) the driveshaft core 46 such that the inner radial layer circumferentially surrounds or covers at least a longitudinal portion of the driveshaft core. For example, the inner radial layer 50 may be extruded directly onto the driveshaft core 46 or the inner radial layer may be reflowed over the core using an outer heat-shrink tube (which may form the outer layer 52, as explained below). The outer radial layer 52 circumferentially surrounds or covers at least a longitudinal portion of the inner radial layer 50, such that the inner radial layer is disposed radially intermediate the driveshaft core 46 and the outer radial layer. As used herein to describe the layers, the terms “inner” and “outer” are relative terms defining the relative locations or positions of the layers with respect to the longitudinal axis LA of the driveshaft 20. It is understood that one or more intermediate radial layers may be formed between the inner and outer radial layers 50, 52, respectively, and one or more layers may be radially outward of the outer radial layer.

Referring to FIG. 7, the driveshaft core 46 comprises multiple filar layers (i.e., 4 radial layers), with each filar layer including a plurality of strands 48 helically wound around the longitudinal axis L of the driveshaft 20. In this embodiment, each of the strands 48 is a core segment of the driveshaft core 46. In particular, the illustrated drive shaft core 46 includes a radially innermost filar layer, generally indicated at 46a, comprising a plurality of helically wound strands (e.g., 3 strands); a first intermediate filar layer, generally indicated at 46b, comprising a plurality of strands helically wound around the radially innermost filar layer (i.e., 9 strands); a second intermediate filar layer, generally indicated at 46c, comprising a plurality of strands helically wound around the first intermediate layer (i.e., 10 strands); and an outer filar layer, generally indicated at 46d, comprising a plurality of strands helically wound around the second intermediate filar layer (i.e., 10 strands). The filar layers 46a, 46b, 46c, 46d are counter-wound relative to radially adjacent filar layer(s), so that the radially innermost filar layer 46a and the second intermediate filar layer 46c are wound in the same direction (e.g., clockwise), and the first intermediate filar layer 46b and the outer filar layer 46d are wound in the same direction that is opposite that of the radially innermost filar layer and the first intermediate filar layer (e.g., counterclockwise). The strands 48 of each filar layer 46a, 46b, 46c, 46d may be wires made from stainless steel, tungsten, or other material. The diameter of the driveshaft core 46 may have any suitable diameter such as from about 0.015 in (0.038 cm) to about 0.040 in (0.10 cm), and in one embodiment, about 0.020 in (0.050 cm).

The driveshaft core 46 may be of other configurations. For example, in other embodiments within the scope of the present invention, the driveshaft core 46 may comprise at least one layer of braided strands, with each strand being a core segment of the driveshaft core. In yet other embodiments, the driveshaft core 46 or the outer layer of the core may comprise at single coiled strand, with each turn of the strand being a core segment or the driveshaft core. In yet other embodiments, the driveshaft core 46 may comprise at least one slit tube having one or more slits (e.g., spaced apart radial slits or a helical slit) to allow for bending along the length of the tube, with each portion adjacent the one or more slits being a core segment of the driveshaft core.

To explain the forces involved when a helically wound or coiled torque-transmitting member (e.g., the driveshaft 36), among other torque-transmitting members, is bent along an arc (see, FIG. 5) and then rotated about its axis LA, schematic representations of fragmentary cross sections of straight and bent longitudinal portions of the illustrated driveshaft are illustrated in FIGS. 8-11. FIGS. 8 and 9 are schematic cross-sectional views of the driveshaft 20 when the driveshaft is in a straight, unloaded configuration, such as shown in FIG. 4. In the illustrated embodiment, bridge portions 60 of the inner radial layer 50 bridge gaps, indicated by Gᵢₙᵢₜᵢₐₗ in FIGS. 8 and 9, between adjacent strands 48 of the outer filar layer 46d of the driveshaft core 46 (only one gap Gᵢₙᵢₜᵢₐₗ is shown in each of FIGS. 8 and 9 for ease of illustration). In this illustrated embodiment, each gap Gᵢₙᵢₜᵢₐₗ is defined between center points C of the adjacent strands 48. FIG. 8 depicts adjacent strands 48 and bridge portions 60 therebetween at an upper portion of the driveshaft 20 when the driveshaft is in its straight and unloaded configuration (the remaining strands are not shown), such as illustrated in FIG. 4, and FIG. 9 depicts adjacent strands and bridge portions therebetween at a lower portion of the driveshaft when the driveshaft is in its straight and unloaded configuration (the remaining strands are not shown). When the driveshaft 20 is in its straight, unloaded configuration, the bridge portions 60 are also in a substantially unloaded state, meaning tensile stresses are not being imparted to the bridge portions by the strands 48. In other embodiments, the gap G may be defined between adjacent turns of a coil, for example, or between any other adjacent portions of a driveshaft core that move relative to one another when the driveshaft is bent along an arc.

The gaps G between adjacent strands 48 of the outer filar layer 46d increase (and decrease) when the driveshaft is bent along an arc, such as when the catheter is moving through or disposed within a tortuous path in a body lumen. Turning to FIGS. 10 and 11, when the longitudinal portion of the coiled driveshaft 20 is bent along an arc, such as an arc having a radius of curvature r shown in FIG. 5, the adjacent strands 48 at the upper portion, which are now at an outer radius of the arc, are urged away from one another, thereby creating a gap G_{bent} between the strands that is larger than the gap Gᵢₙᵢₜᵢₐₗ (i.e., the gap G increases). Simultaneously, the adjacent strands 48 at the lower portion of the driveshaft 20, which are now at an inner radius of the arc, are urged toward one another, which may or may not decrease the gap G (i.e., G_{bent} may or may not be less than Gᵢₙᵢₜᵢₐₗ). Because the bridge portions 60 of the inner radial layer 50 are adhered to the strands 48, increasing the gap G between the strands at the outer radius of the arch induces tensile stress in the corresponding bridge portion(s), causing deformation or strain (e.g., elongation) of the bridge portion(s). At the inner radius of the arc, the urging of the adjacent strands 48 toward one another (i.e., imparting a compressive force) may, in some embodiments, induce compressive stress in the respective bridge portions 60 of the inner layer 50, causing deformation or strain (e.g., contraction) of the bridge portions. In at least one embodiment, the strain of the bridge portion 60 between the strands 48 at the inner radius of the arc is nominal because the gaps G between the adjacent strands are relatively small in the straight, unloaded stage of the driveshaft 20, and therefore, the compressive strain of the bridge portion is restricted.

When the bent portion of the driveshaft 20 is rotated 180 degrees about its axis LA, the adjacent strands 48 that were at the outer radius of the arc (as shown in FIG. 10) move to the inner radius of the arc (see FIG. 11), and the adjacent strands that were at the inner radius of the arc (as shown in FIG. 11) move to the outer radius of the arc (see FIG. 10). Accordingly, as corresponding adjacent strands 48 rotate toward the inner radius of the arc, a compressive force is applied to bridge portions 60 of the inner layer 50 at the corresponding adjacent strands. Simultaneously, as the corresponding adjacent strands 48 rotate toward the outer radius of the arc, a tensile force is applied to the bridge portions 60 of the inner layer 50 at the corresponding adjacent strands due to the adjacent strands moving apart (increasing the gap G_{bent}) at the outer radius of the arc. The tensile force induces tensile stress in the bridge portions 60, causing tensile strain based on the gap change ΔG calculated by taking the difference between the gap G_{bent} when the driveshaft 20 is bent and the gap Gᵢₙᵢₜᵢₐₗ when the driveshaft is straight and unloaded (i.e., ΔG=G_{bent}−Gᵢₙᵢₜᵢₐₗ).

As can be understood, if the inner layer 50 does not have a suitable elastic yield strain, as determined at least in part by the gap change ΔG when the driveshaft 20 is bent along an arc, then the bridge portions 60 at the outer radius of the arc (as shown in FIG. 10) of the bent driveshaft will undergo plastic deformation (i.e., plastic elongation). After undergoing plastic deformation, the bridge portions 60 at the outer radius of the arc will resist movement back to their initial length. This may be problematic because as the driveshaft 20 rotates and the bridge portion(s) 60 between the adjacent strands 48 at the outer radius of the arc that underwent plastic deformation rotate to the inner radius of the arc, the plastically deformed bridge portion(s) will resist movement of the corresponding adjacent strands toward and away from one another at the inner radius of the arc. Due to this resistance of the plastically deformed bridge portion(s) 60 to deform back to its initial unloaded length under compression and to elongate during tension, the bent driveshaft 20 resists rotation about its axis LA.

According to the teachings of the present disclosure, to inhibit plastic deformation of the bridge portions 60 of the inner layer 50 due to the gap change ΔG between adjacent strands 48, a material (e.g., a polymer) used to form the inner layer of a particular driveshaft 20 is chosen based on the elastic yield strain of the material and the potential gap change between adjacent strands experienced when the driveshaft is bent along an arc having a selected minimum radius of curvature and rotated about its rotational axis. For example, if adjacent strands experience a 0.1 mm change in gap ΔG when the driveshaft is bent at a predetermined arc (e.g., 0.5 in ((1.27 cm)) and the initial gap is 0.5 mm, then the material chosen to form the inner layer should have a minimum elastic yield strain of 20% so that the inner layer does not, at least theoretically, undergo plastic deformation when the driveshaft is bent at the predetermined arc having a minimum radius of curvature.

In the illustrated example, the inner radial layer 50 comprises an elastomer, such as silicone, latex, and thermoplastic elastomers, among others. Suitable thermoplastic elastomers include styrenic block copolymers, polyolefin blends, elastomeric alloys (TPE-v or TPV), thermoplastic polyurethanes, thermoplastic copolyester, and thermoplastic polyamides. Suitable commercially available thermoplastic elastomers derived from block copolymers include ARNITEL® (available from DSM), ENGAGE® (available from The Dow Chemical Company), HYTREL® (available from DUPONT) DRYFLEX® and MEDIPRENE® (available from ELASTO), KRATON® (available from Shell chemical division), and PEBAX® (available from Arkema), which is a PEBA (polyether block amide) thermoplastic elastomer.

In the illustrated example, the elastomer (e.g., thermoplastic elastomer) of the inner radial layer 50 of the driveshaft 20 has an elastic yield strain from at least about 25%, or from at least about 50%, or from at least about 100%, or from at least about 150%, or from at least about 200%. For example, the inner radial layer 50 may have an elastic yield strain of from about 25% to about 2000%, or from about 25% to about 1000%, or from about 25% to about 500%, or from about 25% to about 300%, or from about 25% to about 200%, or from about 25% to about 150%, or from about 25% to about 100%, or from about 50% to about 2000%, or from about 50% to about 1000%, or from about 50% to about 500%, or from about 50% to about 200%, or from about 50% to about 150%, or from about 75% to about 2000%, or from about 75% to about 1000%, or from about 75% to about 500%, or from about 75% to about 200%, or from about 75% to about 100%, or from about 100% to about 2000%, or from about 100% to about 500%, or from about 150% to about 2000%, or from about 150% to about 500%.

It may also be beneficial for the elastomer (e.g., thermoplastic elastomer) of the inner radial layer 50 to have, in addition to a suitable elastic yield strain, a low modulus of elasticity relative to the outer radial layer 52. For example, in the illustrated example the ratio of the modulus of elasticity of outer radial layer 52 to the inner radial layer may be from at least about 1.5:1, or from at least about 2:1, or from at least about 5:1, or from at least about 10:1, or from at least about 20:1, or from at least about 50:1. In one example, the ratio of the modulus of elasticity of outer radial layer 52 to the inner radial layer may be from 1.5:1 to about 50:1, or from about 2:1 to about 50:1, or from about 1.5:1 to about 25:1. In this way, the transmission of tensile strain (i.e., elongation) of the bridge portions 60 to the outer radial layer 52 is reduced because the bridge portions will readily deform at the interface of the inner and outer radial layers without causing significant strain (i.e., elongation) of the outer layer at the interface. Moreover, by having a low modulus of elasticity, the driveshaft 20 is more axially flexible and more readily bendable into an arc. The modulus of elasticity of the inner radial layer 50 may be from about 5 MPa to about 100 MPa, or from about 5 MPa to about 50 MPa, or from about 5 MPa to about 20 MPa, or from about 5 MPa to about 10 MPa.

In the illustrated example, the outer radial layer 52 applies a radially compressive force to the inner radial layer 50 and the driveshaft core 46 to retain the strands 48 in their respective orientations relative to one another and inhibit shifting of the strands. Also in the illustrated example, the outer radial layer 52 has a low coefficient of friction (i.e., has a high lubricity) and high abrasion-resistance so as to respectively inhibit energy loss in the form of friction and inhibit deterioration of the outer layer when the driveshaft 20 rubs against the wall of the body lumen 16 as the driveshaft rotates. Suitable material for the outer radial layer 52 includes, for example, PTFE, FEP, or Polyolefin, among others. As disclosed above, the outer radial layer 52 may comprise a heat-shrink tube that is heat-shrunk over the inner radial layer 50. The heat-shrink tube binds onto the inner radial layer 50 with sufficient radial force so that the outer radial layer does not move or shift longitudinally relative to the inner radial layer.

As disclosed above, in the illustrated example the outer radial layer 52 of the driveshaft 20 may have a modulus of elasticity that is greater than the modulus of elasticity of the inner radial layer 50. Again, this configuration reduces the transmission of strain from the inner radial layer 50 to the outer radial layer 52. Moreover, by having a greater modulus of elasticity, the outer radial layer 52 maintains the driveshaft core 46 (e.g., the strands 48) in its proper configuration (e.g., maintains the strands in proper arrangement relative to one another). Without a suitable outer radial layer having a sufficiently high modulus of elasticity, the strands 48 of the driveshaft core 46 may shift relative to one another and become disorganized, which may lead to buckling of the driveshaft 20.

Although the outer radial layer 52 does not experience the strain (or at least a significant portion thereof) imparted by the gap change ΔG between adjacent strands 48, the outer radial layer will experience some strain when the driveshaft 20 is bent along an arc. Accordingly, to inhibit plastic deformation of the outer radial layer 52, the material of the outer radial layer may have an elastic yield strain greater than one half the ratio of the diameter of the laminated driveshaft 20 divided by the minimum radius of curvature to which the driveshaft will be subjected. For example, with a 0.025 in (0.064 cm) diameter driveshaft subjected to minimum of 0.25 in (0.64 cm) radius of curvature, the yield strain of the outer radial layer 52 should be greater than 0.025/0.25/2=5%. In other examples, the elastic yield strain of the outer layer may be from about 2% to about 50%, or from about 2% to about 20%, or from about 2% to about 10%, or from about 5% to about 20%.

Because in the illustrated embodiment the driveshaft 20 is received in the lumen 16 of the catheter body 12 and rotates therein, the outer radial layer (which is the outermost layer in the illustrated embodiment) may have an outer surface having a high lubricity and high abrasion-resistance. The high lubricity reduces energy lost due to friction between the rotating driveshaft 20 and the catheter body 12. As an example, the outer radial layer (or another outmost radial layer) may have a low coefficient of friction, such as from about 0 to about 0.25, or from about 0.05 to about 0.1.

### Experimental Example

The internal torsional resistance of a driveshaft constructed according to the principles of the present invention was measured and compared to the internal torsional resistance of drive shafts constructed according to conventional methods. The internal torsional resistance is the resistant force that opposes rotation of a driveshaft about its longitudinal axis. Each of four tested drive shafts had identical driveshaft cores (i.e., a 0.020 in (0.051 cm)) diameter driveshaft core comprising 304V stainless steel wires arranged in four filar layers. An innermost filar layer included a single wire running along the longitudinal axis; an inner intermediate filar layer included six wires helically wound in a first direction around the innermost wire; an outer intermediate filar layer including nine wires helically wound in a second direction around the inner intermediate layer; and an outermost filar layer including twelve wires helically wound in the first direction around the outer intermediate layer. The four tested drive shafts had the following layers over the core: i) no layers; ii) inner radial layer of PEBAX® 3533 and an outer radial layer of PTFE (the “PEBAX®/PTFE driveshaft”); iii) a single radial layer of Grilamid L25 (an extrudable, nylon 12 material) having a thickness of 0.003 in (0.008 cm) (the “thick Grilamid L25 driveshaft”); and iv) a single radial layer of Grilamid L25 having a thickness of 0.001 in (0.003 cm) (the “thin Grilamid L25 driveshaft”).

The PEBAX®/PTFE driveshaft was constructed according to the principles of the present invention, using a reflow process. It was determined that when the driveshaft was bent along an arc having a radius of curvature r of about 0.5 in (1.27 cm), the strands at the outer radius of the arc experience a gap change percentage (% ΔG) of about 75%. The gap change percentage (% ΔG) was calculated by measuring (or estimating) the gap change (ΔG) and dividing that value by the initial gap measurement (Gᵢₙᵢₜᵢₐₗ). This calculation is represented by following formula:

% ΔG=((G_{bent}−Gᵢₙᵢₜᵢₐₗ)/Gᵢₙᵢₜᵢₐₗ)×100

PEBAX® 3533 was selected as the inner layer based on the calculated gap change percentage (% ΔG) of about 75%. FIG. 12 is a graph of the elastic yield strains (or elastomeric limits) of certain PEBAX® polymers. As shown in the graph, PEBAX® 3533 has an elastomeric limit of about 90%. Accordingly, PEBAX® 3533 has an elastic yield strain greater than the localized tensile strain that the bridge portions of the inner layer undergo when the driveshaft is bent along an arc having a radius of curvature r of about 0.5 in (1.27 cm) (hereinafter, the “selected arc”). Therefore, the PEBAX® 3533 bridge portions should not undergo plastic deformation when the driveshaft is bent along the selected arc. It is noted that according FIG. 12, PEBAX® 2533 may also be suitable for the inner radial layer of the driveshaft because it has an elastic yield strain of about 130%. Other elastomeric material may also be suitable for the inner radial layer. PEBAX® 3533 also has a low modulus of elasticity of 12 MPa, which as set forth above, inhibits transmission of strain to the outer radial layer.

A PTFE heat-shrink tube was selected as the outer radial layer because of its low coefficient of friction (i.e., its lubricity), its abrasive-resistance, its ability to contract to a relatively small diameter to apply a suitable radial compressive force to retain the organization of the driveshaft strands, and its elastic yield strain being sufficient to inhibit plastic deformation when the driveshaft is bent along the selected arc. The selected PTFE heat-shrink tube was configured to have a 0.040 in (0.102 cm) expanded inner diameter, a 0.015 in (0.038 cm) recovered (i.e., contracted) inner diameter, and a wall thickness of 0.003 in (0.008 cm) when recovered. The PTFE heat-shrink tube had a coefficient of friction from 0.04 to 0.1.

In forming the driveshaft, a sleeve formed from PEBAX® 3533 was provided, and the driveshaft core was inserted into the sleeve. The PEBAX® 3533 sleeve had an inner diameter measuring 0.021 in (0.533 mm) +/−0.001 in (0.0254 mm), and a wall thickness of 0.0012 in (0.003 cm)+/−0.0003 in (0.0008 cm). The PTFE heat-shrink tube was sleeved over the PEBAX® sleeve that was received on the driveshaft core, and the driveshaft assembly was reflowed using a hot box. After reflowing, the outer diameter of the PEBAX®/PTFE driveshaft measured from about 0.0248 in (0.0630 cm) to about 0.0252 in (0.0640 cm). Accordingly, the thickness of the PEBAX®/PTFE laminate was from about 0.0024 in (0.0610 mm) to about 0.0026 in (0.0660 mm).

The two Grilamid L25 drive shafts were constructed according to conventional methods, including reflowing the Grilamid L25 layer using a large upright over and an FEP heat-shrink tube. The FEP heat-shrink tube was removed after reflowing. Grilimad L25 has an elastic yield strain of 6%, and a high tensile modulus of 110 MPa. Theoretically, an elastic yield strain of 6% should be sufficient to inhibit plastic deformation due solely to the driveshaft being bent along the arc, without accounting for the strain caused by adjacent core segments moving apart. In other words, Grilimad L25 has elastic yield strain (i.e., 6%) greater than one half the ratio of the diameter of the laminated driveshaft 20 divided by the minimum radius of curvature to which the driveshaft will be subjected.

Referring to FIG. 13, a Torque Resistance Testing Apparatus, generally indicated at 100, including a Torque Test Fixture, generally indicated at 102, and a Torsional Resistance Fixture, generally indicated at 104, was used to determine the internal torsional resistances of the drive shafts. The Torque Test Fixture 102 includes a torque gage 106 that measures the internal torsional resistance imparted on a chuck 108 that is adapted to hold a proximal end of the driveshaft portion, a gage readout 110 in communication with the load cell for displaying torque imparted on the chuck, and a torque gage mount 111 secured to a board 120. The Internal torsional resistance Fixture 104 includes a plate 112 with a curved tube 114 having a curved portion 114a with a radius of curvature of about 0.5 in (1.27 cm), and a handle 116 to allow the user to rotate the plate.

To set-up the test, the following steps were performed for each of the driveshafts:
1. ensure that the torque gage 106 of the Torque Test Fixture 102 is 10 oz-in (7.1 N-cm) capacity;
2. place the Internal torsional resistance Fixture 104 on a board 120 to which the Torque Test Fixture 102 is secured; the internal torsional resistance Fixture 104 should be spaced a distance d₁ of approximately 7 in (17.8 cm) from the chuck 108; tighten a thumbscrew 122 to secure the Internal torsional resistance Fixture 104 to the board 120;
3. verify torque gage thumbscrew 122 securing the torque gage 106 to the mount 111 is tightened so the torque gage cannot move relative to the mount;
4. adjust the torque gage mount 111 so that there is a 0.5 in (1.27 cm) distance d₂ (FIG. 14) between the chuck 108 and the path plate 112 then tighten mount thumbscrew 128;
5. using a ruler and wire cutters, cut driveshaft samples to be tested to a length of 7.25 in (18.4);
6. using a sharpie marker, mark one end of each sample to identify which end will be chucked; and
7. if tracing individual samples is desired, place each sample in its own bag and write the sample number on the bag.

The following steps are steps taken to perform the test:
1. insert driveshaft sample into the tube 114 on the plate 112 until the free end of the driveshaft is flush with the edge of the path plate;
2. tighten the chuck 108 onto the test driveshaft to apply roughly 9 oz-in (6.4 N-cm) of torque without exceeding the 10 oz-in (7.1 N-cm) capacity of the torque gage, and verify the sample is in the center of the chuck;
3. rapidly spin the Internal torsional resistance Fixture handle 116 clockwise until the torque gage display 110 shows a stable reading;
4. record the internal torsional resistance measurement;
5. remove sample driveshaft from the chuck 108 and the Internal torsional resistance Fixture 104; and
6. repeat Steps 1 through 5 for the remainder of the test drive shafts.

Table I (below) shows the results of the testing. A graph showing these results of the testing is presented in FIG. 15.

TABLE I
Results of Testing

| | | | | | | Internal |
|---|---|---|---|---|---|---|
| | | | | | | torsional |
| | | | | | Heat- | resistance |
| Std | Run | Group | Coil | Laminate | shrink | (oz-in) |
| 1 | 1 | 1 | SS304V | Thin L25 | Large FEP | 0.15 |
| 2 | 17 | 1 | SS304V | Thin L25 | Large FEP | 0.15 |
| 3 | 33 | 1 | SS304V | Thin L25 | Large FEP | 0.15 |
| 4 | 2 | 2 | SS304V | Thick L25 | Large FEP | 0.21 |
| 5 | 18 | 2 | SS304V | Thick L25 | Large FEP | 0.21 |
| 6 | 34 | 2 | SS304V | Thick L25 | Large FEP | 0.22 |
| 7 | 3 | 3 | SS304V | Thin L25 | Small FEP | 0.15 |
| 8 | 19 | 3 | SS304V | Thin L25 | Small FEP | 0.16 |
| 9 | 35 | 3 | SS304V | Thin L25 | Small FEP | 0.15 |
| 10 | 4 | 4 | SS304V | Thick L25 | Small FEP | 0.22 |
| 11 | 20 | 4 | S5304V | Thick L25 | Small FEP | 0.22 |
| 12 | 36 | 4 | SS304V | Thick L25 | Small FEP | 0.20 |
| 13 | 5 | 5 | SS304V | PEBAX ® 25D | PTFE | 0.07 |
| 14 | 21 | 5 | SS304V | PEBAX ® 25D | PTFE | 0.05 |
| 15 | 37 | 5 | SS304V | PEBAX ® 25D | PTFE | 0.07 |
| 25 | 9 | 9 | SS304V | None | N/A | 0.01 |
| 26 | 25 | 9 | SS304V | None | N/A | 0.01 |
| 27 | 41 | 9 | SS304V | None | N/A | 0.01 |

As can be seen from Table I and FIG. 15, the internal torsional resistance of the PEBAX®/PTFE driveshaft was less than 0.10 oz-in (0.07 N-cm) and less than the internal torsional resistance of the thin and thick Grilamid L25 drive shafts. Only the driveshaft that included no radial layers had an internal torsional resistance less than PEBAX®PTFE driveshaft, which was to be expected since there was no material inhibiting rotation of this driveshaft and the only energy lose may be due to frictional resistance between filar layers. It is believed that the internal torsional resistance of the PEBAX®/PTFE driveshaft was due to the fact that the elastic yield strain of the PEBAX® 3533 (i.e., about 90%) is greater than localized strain of the bridge portions between adjacent strands caused by the change in gap percentage % ΔG (i.e., about 75%) when the driveshaft is bent along the arc. Moreover, it is believed that the bridge portions may remain completely attached to the filars (i.e., the bridge portions will not partially detach), thereby causing additional strain of the bridge portions. It is further believed that the internal torsional resistances of the thin and thick Grilamid L25 drive shafts were 2 to 4 times greater than the internal torsional resistance of the PEBAX®/PTFE driveshaft because the elastic yield strain of Grilamid L25 (i.e., about 6%) is less than localized strain of the bridge portions between adjacent strands caused by the change in gap percentage % AG (i.e., about 75%) between the adjacent strands at the outer radius of the arc.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements. In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A catheter comprising:
a flexible elongate body defining a lumen extending longitudinally within the body;
a flexible elongate torque-transmitting member received in the lumen of the body, the torque-transmitting member having proximal and distal end portions and configured to transmit torque applied to the proximal end portion toward the distal end portion, the torque-transmitting member including
a structural core having a plurality of adjacent core segments, wherein the structural core is configured such that bending of the torque-transmitting member along an arc urges the core segments at an outer radius of the arc away from one another, said plurality of core segments defining an exterior surface of the core,
an inner radial layer covering a longitudinal portion of the exterior surface of the core and having an inner surface secured to the plurality of core segments such that the inner layer experiences localized tensile strain when the core segments are urged away from one another during bending of the torque-transmitting member, wherein the inner layer comprises an elastomer, the inner layer having an exterior surface, and
an outer radial layer circumferentially covering a portion of the exterior surface of the inner layer such that the inner layer is radially intermediate the structural core and the outer layer; and
a functional element operatively coupled to the torque-transmitting member for rotation relative to the body.

2. The catheter of claim 1, wherein the inner layer has an elastic yield strain greater than the localized tensile strain of the inner layer between corresponding adjacent core segments when the torque-transmitting member is bent along an arc having a radius of curvature less than or equal to about 0.5 in (1.27 cm).

3. The catheter of claim 1, wherein the inner layer has an elastic yield strain greater than about 25%.

4. The catheter of claim 3, wherein the inner layer has an elastic yield strain from about 50% to about 150%.

5. The catheter of claim 1, wherein the inner layer has a modulus of elasticity from about 5 MPa to about 100 MPa.

6. The catheter of claim 5, wherein the inner layer has a modulus of elasticity from about 5 MPa to about 20 MPa.

7. The catheter of claim 1, wherein the elastomer is a thermoplastic elastomer.

8. The catheter of claim 7, wherein the thermoplastic elastomer is a PEBA thermoplastic elastomer.

9. The catheter of claim 1, wherein the ratio of a modulus of elasticity of the outer radial layer to the inner radial layer is from about 1.5:1 to about 10:1.

10. The catheter of claim 1, wherein the outer radial layer comprises heat-shrink tube, wherein the heat-shrink tube is heat shrunk around the inner radial layer.

11. The catheter of claim 10, wherein the heat-shrink tube comprises one of PTFE, FEP, and Polyolefin.

12. The catheter of claim 11, wherein the heat-shrink tube has a coefficient of friction from about 0 to about 0.25.

13. The catheter of claim 1, wherein the torque-transmitting member has an internal torsional resistance from about 0.05 in-oz (0.05 N-cm) to about 0.10 oz-in (0.07 N-cm).

14. The catheter of claim 1, wherein the structural core comprises a filar layer including plurality of strands wound around the longitudinal axis in a helix, wherein each strand of the filar layer forms one of the core segments.

15. The catheter of claim 1, wherein the structural core comprises a filar layer consisting of a single strand wound around the longitudinal axis in a helix, wherein each turn of the single strand forms one of the core segments.

16. A catheter comprising:
a flexible elongate body defining a lumen extending longitudinally within the body;
a flexible elongate torque-transmitting member received in the lumen of the body, the torque-transmitting member having proximal and distal end portions and a longitudinal axis and configured to transmit torque applied to the proximal end portion toward the distal end portion, the torque-transmitting member including
a flexible structural core having a plurality of adjacent longitudinal core segments, wherein the structural core is configured such that bending of the torque-transmitting member along an arc urges the core segments at an outer radius of the arc away from one another, said plurality of core segments defining an exterior surface of the core,
an inner radial layer circumferentially covering at least a longitudinal portion of the exterior surface of the core and having an inner surface secured to the plurality of core segments such that the inner layer experiences localized tensile strain when the core segments are urged away from one another during bending of the torque-transmitting member, wherein the inner layer comprises an elastomer, the inner layer having an exterior surface, and
an outer radial layer circumferentially covering at least a portion of the exterior surface of the inner layer such that the inner layer is radially intermediate the structural core and the outer layer,
wherein the torque-transmitting member has an internal torsional resistance from about 0.05 in-oz (0.04 N-cm) to about 0.10 oz-in (0.07 N-cm) when bent along an arc having a radius of curvature of about 0.5 in (1.27 cm); and
a functional element operatively coupled to the torque-transmitting member for rotation relative to the body.

17. The catheter of claim 16, wherein the inner radial layer has an elastic yield strain greater than the localized tensile strain of the inner layer between corresponding adjacent core segments when the torque-transmitting member is bent along an arc having a radius of curvature less than or equal to about 0.5 in (1.27 cm), the inner layer having an exterior surface.

18. The catheter of claim 16, wherein the outer radial layer comprises heat-shrink tube, wherein the heat-shrink tube is heat shrunk around the inner radial layer.

19. A catheter comprising:
a flexible elongate body defining a lumen extending longitudinally within the body;
a flexible elongate torque-transmitting member received in the lumen of the body, the torque-transmitting member having proximal and distal end portions and a longitudinal axis and configured to transmit torque applied to the proximal end portion toward the distal end portion, the torque-transmitting member including
a flexible structural core having a plurality of adjacent longitudinal core segments, wherein the structural core is configured such that bending of the torque-transmitting member along an arc urges the core segments at an outer radius of the arc away from one another, said plurality of core segments defining an exterior surface of the core,
an inner radial layer circumferentially covering at least a longitudinal portion of the exterior surface of the core and having an inner surface secured to the plurality of core segments such that the inner layer experiences localized tensile strain when the core segments are urged away from one another during bending of the torque-transmitting member, wherein the inner layer comprises a thermoplastic elastomer having an elastic yield strain from at least about 50%, and
an outer radial layer circumferentially covering at least a longitudinal portion of the exterior surface of the inner layer such that the inner layer is radially intermediate the structural core and the outer layer, wherein the outer radial layer comprises a heat-shrink tube; and
a functional element operatively coupled to the torque-transmitting member for rotation relative to the body.

20. The catheter of claim 19, wherein the torque-transmitting member has an internal torsional resistance from about 0.05 in-oz (0.04 N-cm) to about 0.10 oz-in (0.07 N-cm) when bent along an arc having a radius of curvature of about 0.5 in (1.27 cm).
